(19) United States
(12) Patent Application Publication
(10) Pub. No.: US 2005/0197548 A1
(43) Pub. Date: Sep. 8, 2005

(54) DISPOSABLE/REUSABLE FLEXIBLE SENSOR

(75) Inventor: Thomas Dietiker, Rancho Palos Verdes, CA (US)

Correspondence Address: FACTOR & LAKE, LTD, 1327 W. WASHINGTON BLVD., SUITE 5G/H, CHICAGO, IL, 60607, US

(73) Assignee: ELEKON INDUSTRIES USA, INC., Torrance, CA (US)

(21) Appl. No.: 10/708,476

(22) Filed: Mar. 5, 2004

(51) Int. Cl. A61B 5/00

(52) U.S. Cl. 600/323

(57) ABSTRACT

The present invention is directed to an improved pulse oximetry sensor device, in which the top and bottom portions of the sensor housing are affixed at a crease point, and are foldable towards each other to create an enclosure therebetween for retaining the optical elements of the device. The sensor housing may then be affixed to a patient by any number of methods, including using adhesive on a flexible strap, or by using a Velcro® strap on a flexible strap. Additionally discussed is a method of manufacturing such a device, and methods to reduce the manufacturing costs of an otherwise disposable device.

### BACKGROUND OF INVENTION

1. General Field of the Invention

The present invention relates generally to devices for the non-invasive measurement of physiologic conditions, such as oxygen content of the blood through non-invasive pulse oximetry. Specifically, the present invention relates generally to devices for such measurement, and methods of manufacturing those devices.

2. Background of the Invention

Noninvasive pulse oximetry is a well known technology, providing a wide range of devices in the art. Typically, such devices operate on the principles of light absorption by oxygenated and unoxygenated hemoglobin. By passing a known wavelength of light through the translucent tissues of a patient, and measuring the absorption of that light for a period of time, the oxygen content of the blood passing through that tissue can be measured.

Although numerous devices are known in the art, there are still significant issues with economics of manufacture and ease of operation.

It is therefore an object of this invention to provide an improved device with easier operation and manufacture.

It is additionally an object of this invention to provide an improved method of manufacturing such a device, and a method for ensuring continued economically conscious use of that device.

These and other objects will become apparent to one of ordinary skill in the art in light of the specification, claims and drawings appended hereto.

### SUMMARY OF INVENTION

The present invention is directed to an improved sensor housing for measuring light transmission across a tissue of a patient. The sensor housing includes a top member and a bottom member formed in substantially identical, predetermined shapes, and which are joined at a crease point. The top and bottom members may be folded towards each other at the crease point to, in turn, create an enclosure therebetween, which is configured to receive an emitter and a detector for transilluminating the tissues of a patient. To transmit and receive the light, the top member has a first and a second aperture therein, and the emitter and detector are substantially optically aligned with these apertures such that when the sensor housing is flexed into operative position, the first and second apertures are in substantial optical alignment.

The sensor housing is preferably manufactured from a flexible material, which may additionally be opaque, and have a low Shore hardness. Generally such materials will not slip adjacent the skin of a patient. Additionally, the sensor housing may additionally include a top surface that includes a raised portion that is curvilinear to cooperate with a finger of a patient, or may include a portion that is manufactured from a malleable material capable of substantially molding itself to the shape of a patient's tissues.

Preferably, the sensor housing is sealed against intrusion of one or more of foreign bodies, moisture and ambient light. Therefore, the sensor housing preferably includes a sealing means along its periphery, such as an adhesive seal, an ultrasonic or heat welded seal or similar means.

The emitter and detector are connected together using wiring or other electrical connection means, and then to an outside measurement device through a wiring device. Preferably, the emitter and detector are both positioned adjacent the top of the sensor housing. Alternatively, both the detector and the emitter may actually extend out of the housing itself. To further enhance operation, the detector may have a conductive material on its rear side, such as copper, to prevent electromagnetic interference.

Such a sensor housing is preferably associated with a transillumination device to facilitate attachment to a patient. The device generally includes a backing substrate to which the other elements are attached, a structure for affixing the sensor housing to the backing substrate, and means for attaching the trans-illumination device to a patient.

In one embodiment, the sensor housing is affixed to the backing structure using a flexible strap. The flexible strap may be butterfly in shape if desired, or a simple long strap of flexible material. If a strap is used, the sensor housing may include a flange to affix itself more securely to the strap. Alternatively, the sensor housing may be affixed by one or more brackets. Preferably, the sensor housing extends beyond and above the surface of the strap or bracket to contact the skin of the patient.

The device may then be attached to patient by using an adhesive material on the surface of one or more of the strap, the backing, the sensor housing or the brackets. Alternatively, if a wrap-around strap is used, a Velcro® strap can be associated with the strap to ensure attachment to a patient.

The present invention is also directed to a method of manufacturing a sensor housing for a transillumination device, wherein the method includes the steps of (1) molding a top member and a bottom member from a unitary piece of material, wherein the top and bottom members include a crease point, and the top member includes a first and a second aperture; (2) inserting an emitter and a detector into at least one of the top and bottom members so that they are in substantial optical alignment with the first and second aperture respectively; (3) connecting the emitter and detector together with an electrical connection; (4) securing the emitter and detector in the at least one top and bottom member; and (5) folding the top and bottom members at the crease point to, in turn, form an enclosure therebetween. Preferably the method also includes the step of sealing the enclosure around a periphery of the top and bottom members by one or more of welding, or adhesive sealing.

The present invention is further directed to a method for remanufacturing an otherwise disposable transillumination device, including the steps of (1) acquiring an otherwise disposable device, the device comprising a sealed sensor housing connected to a wiring device, and an attachment member for attaching the sensor housing to a patient; (2) removing the attachment member from the disposable device; (3) sanitizing and or sterilizing the sealed sensor housing and the wiring device; and (4) reassociating the sealed sensor housing with a new attachment member to, in turn, facilitate the use of the device on a patient.

### BRIEF DESCRIPTION OF DRAWINGS

FIG. 1 of the drawings depicts a top view of one preferred embodiment of the invention;

FIG. 2A of the drawings depicts a cut out view of a sensor housing as described in the present invention;

FIG. 2B of the drawings depicts a top view of a sensor housing according to the invention;

FIG. 3 of the drawings depicts an exploded view of a transillumination device according to one embodiment of the present invention;

FIG. 4 of the drawings depicts an exploded view of another embodiment of the present invention;

FIG. 5 of the drawings depicts an exploded view of another embodiment of the present invention; and

FIG. 6 of the drawings depicts an exploded view of another embodiment of the present invention.

### DETAILED DESCRIPTION

While this invention is susceptible of embodiment in many different forms, there is shown in the drawings and will be described in detail, several specific embodiments with the understanding that the present disclosure is to be considered as an exemplification of the principles of the invention and is not intended to limit the invention to the embodiments illustrated.

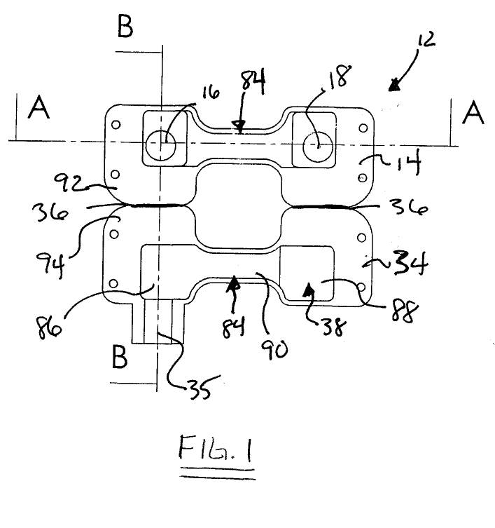

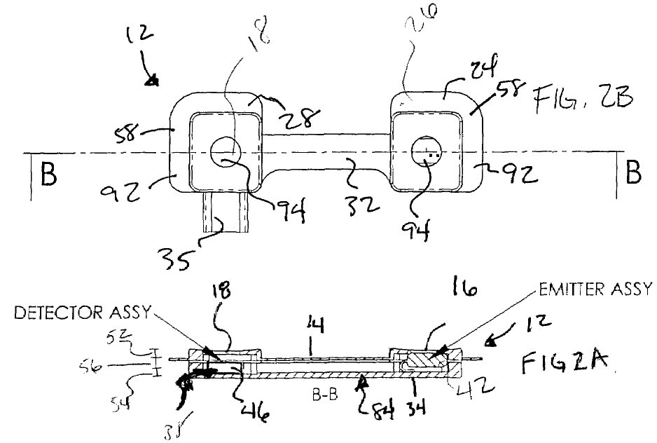

The present invention, as shown in FIG. 1, comprises an improved sensor housing for use in transillumination devices, such as SpO2 pulse oximetry sensors. Sensor housing 12 is shown in its pre-assembly condition as a single piece of molded, flexible material formed into top member 14 and bottom member 34, which are joined together at crease point 36. Top member 14 and bottom member 34 comprise a flexible, opaque material that is formed into substantial mirror image shapes, so that a user may fold top member 14 or bottom member 34 towards the other at crease point 34, forming enclosure 38 (shown best in FIG. 2A) therebetween. Enclosure 38, in turn, provides a housing for retention of one or more optical elements for transilluminating a tissue of a patient.

Crease point 34 is shown in FIG. 1 as comprising the intersection between lateral side 92 of top member 14, and lateral side 94 of bottom member 34. Crease point 36, however, can comprise any single point of intersection between top member 14 and bottom member 34 that will allow the top member 14 and bottom member 34 to overlay each other upon folding at crease point 34. Thus, crease point 36 could comprise a shared side of top member 14 and bottom member 34, or could comprise a single point of attachment, as would be understood by one of ordinary skill in the art.

Once formed, and as shown in FIG. 2A, enclosure 38 is configured to incorporate a light emission source, or emitter 42, and a light detection device, or detector 46 therein. Such devices are well known in the art, with emitter 42 emitting a light of a known wavelength, and detector 46 being capable of measuring the intensity of light of a certain wavelength. Emitter 42 and detector 46 are positioned within enclosure 38, proximate and in optical alignment with first aperture 16 and second aperture 18 in top member 14. Preferably, detector 46 includes a conductive material on its back side, such as copper, to shield detector 46 from electromagnetic interference.

To facilitate the insertion of emitter 42 and detector 46, and as shown in FIG. 1, top member 14 and bottom member 34 include recessed areas 84, including emitter recess 86, detector recess 88 and wiring recess 90. As shown best in FIG. 2A, recessed areas 84 provide a height to enclosure 38, giving it a top portion 52 adjacent top member 14, bottom portion 54 adjacent bottom member 34, and thickness 56 therebetween. This height allows emitter 42 and detector 46 to be located within enclosure 38 for optimum light transmission efficiency. For example, emitter 42 can be placed within top portion 52 of enclosure 38, and adjacent top member 14, to ensure maximum light transmission. Alternatively, emitter 42 can protrude above top portion 52. Detector 46, on the other hand, may be placed within bottom portion 54 far away from second aperture 18, to limit the interference from surrounding ambient light. Of course, other configurations are also possible depending upon the particular application of the device.

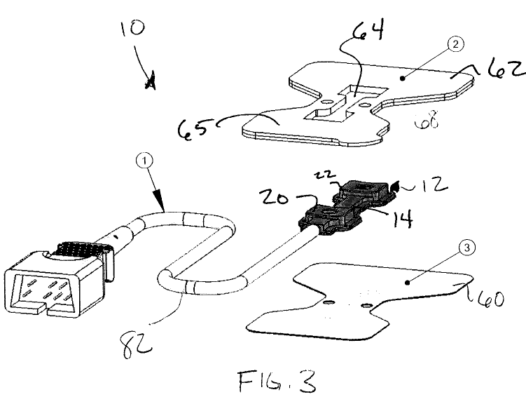

Emitter 42 and detector 46 are connected together via a conventional electrical connection, such as wiring (not shown). Once they are inserted into sensor housing 12, it is sealed along its periphery 92 forming seal 58, as shown in FIG. 2B. Seal 58 may be formed in any number of manners, including by using adhesive, through ultrasonic or heat welding, or other conventional means. Preferably, sensor housing 12 is sealed in its entirely, sealing enclosure 38 and the optical elements contained therein from intrusion from foreign bodies, fluids and/or unwanted ambient light. To fully seal enclosure 38, transparent covering 94 is placed over each of first aperture 16 and second aperture 18. Furthermore, wiring device 82 (see FIG. 3) is inserted into wiring inlet 35 in sensor housing 12, wherein wiring device 82 provides an electrical conduit for connecting emitter 42 and detector 46 to an outside monitoring device, and provides a final seal for enclosure 38 and sensor housing 12.

Sensor housing 12 is constructed from a flexible material such as a polymer or rubberized material, which enables sensor housing 12 to be flexed and placed into operative position on a patient. Preferably, the material has a low Shore hardness, and may be malleable such that the shape of sensor housing 12 conforms substantially to the shape of the tissue to which it is applied. Additionally, although not required, it is preferred that sensor housing 12 be formed of waterproof and/or opaque materials to ensure that water and ambient light can be excluded from enclosure 38. Generally, such materials additionally result in a low slip differential between the finger of the patient and the device.

Operative position for sensor housing 12 is achieved by wrapping sensor housing 12 around a blood-profused area, such as a finger or a nose of a patient, so that first aperture 16 and second aperture 18 are in substantial optical alignment. In this position, light from emitter 42 passes out of first aperture 16, through the tissues of the patient, into second aperture 18, and is received by detector 46.

Depending upon the desired area of application for sensor housing 12, the sensor housing 12 can be formed into any number of shapes. As shown in FIG. 1, to form the shape of sensor housing 12, top member 14 and bottom member 34 have substantially the same shape, helping to facilitate the formation of enclosure 38. Specifically, and as shown in FIG. 2B, in its final folded position sensor housing 12 comprises emitter head 24, detector head 28 and wiring conduit 32. Emitter head 24 and detector head 28 are shown generally as rectangular in shape, but only need to be configured and shaped such that emitter recess 86 and detector recess 88 are sufficient in size and shape to accommodate emitter 42 and detector 46, respectively. Thus, any shape and configuration may be possible.

Wiring conduit 32 may similarly be shaped in a variety of ways, depending upon the application of the device. Wiring conduit 32, however, must be of sufficient length to enable sensor housing 12 to be placed in operative position.

To further facilitate the operative positioning of sensor housing 12, and as best seen in FIG. 3, top member 14 includes top surface 20, which in turn includes a raised portion 22 configured to cooperate with the curvature of a patient's tissue. For example, raised portion 22 can comprise a curvilinear section that is substantially similar to the curvature of a patient's finger, thus cooperating with the finger when in operative position.

Together, sensor housing 12 and wiring device 82 form a complete, reusable device that can, after proper sanitation, be utilized on multiple patients for multiple transillumination measurements. In order to be used, however, sensor housing 12 must be affixed to the patient in operative position. To do so, a user may flex and affix sensor housing 12 in position using an adhesive applied to top surface 20 of sensor housing. Alternatively, medical tape can be wound around sensor housing 12 to affix it in place. Other conventional means may be similarly used, as would be known to one of ordinary skill in the art.

Preferably, sensor housing 12 is utilized within transillumination device 10, an example of which is shown in FIG. 3, which facilitates the association of sensor housing 12 with a patient. Transillumination device 10 includes sensor housing 12 as described above, backing substrate 60 onto which sensor housing 12 is attached, and butterfly strap 62 overlying and affixed to sensor housing 12 and backing substrate 60. Butterfly strap 62 includes aperture 64 through which sensor housing 12 can extend. As with sensor housing 12, backing substrate 60 and butterfly strap 62 are manufactured from flexible materials, for example polyurethane foam, to enable the device to be applied to curved regions of a patient.

Butterfly strap 62 is particularly shaped for placement around such curved areas of a patient, such as the nasal region, or around the tip of a finger. To further facilitate attachment, butterfly strap 62 additionally includes an adhesive on top surface 65 of butterfly strap 62. During storage, therefore, top surface 65 is preferably covered by release liner (not shown) to protect the adhesive quality of top surface 65.

In order to utilize transillumination device 10 shown in FIG. 3, therefore, a user removes the release liner (not shown), and adheres device 10 to the portion of the patient to be transilluminated. Thereafter, wiring device 82 is connected to an external measurement device wherein measurements of, for example, oxygen content of the patient's blood can be taken.

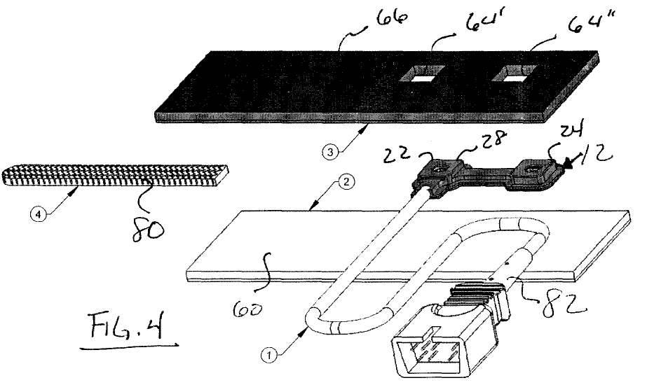

An alternative embodiment of the present invention is shown in FIG. 4, as comprising flexible strap 66 instead of butterfly strap 62. As with butterfly strap 62, flexible strap 66 is manufactured from a medically-compatible flexible material such as polyurethane foam, for conformance with the curvatures of a patient. Flexible strap 66 includes multiple apertures 64′, 64″ that are in turn configured to cooperate with emitter head 24 and detector head 28 of sensor housing 12. Again, sensor housing 12 is affixed to backing substrate 60, and flexible strap 66 is affixed to both sensor housing 12 and backing substrate 60.

The embodiment shown in FIG. 4 is configured to be wrapped around a finger of a patient similar to a medical bandage. To facilitate the attachment, transillumination device 10 includes a Velcro strap 80, and compatible material (not shown) so that device 10 can be applied, and then affixed on a finger by attaching strap 80 to the compatible material, generally associated with backing substrate 60. Of course, other conventional attachment methods could also be used.

Regardless of the particular shape of the material overlaying sensor housing, whether it is butterfly strap 62 or flexible strap 66, it is contemplated that sensor housing 12 extend above and beyond the top surface of the strap, enabling preferred portions of the sensor housing 12, such as raised portion 22, emitter 42 or detector 46, to come into direct contact with a patient's skin, as desired.

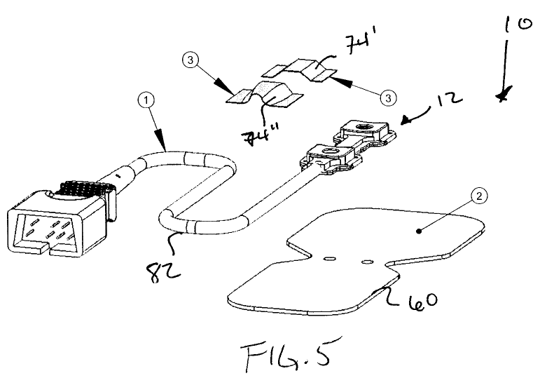

Another alternative embodiment is shown in FIG. 5, wherein straps are replaced with one or more brackets 74′, 74″. Brackets 74′, 74″ are formed from medically-compatible materials that can further secure sensor housing 12 to backing substrate 60, such as, for example, polyurethane tape or the like. Brackets 74′, 74″ are configured to overlay one or more of wiring conduit 32, or wiring device 82, and attach directly to backing substrate 60, to secure sensor housing 12 thereto. To facilitate the attachment, backing substrate 60 is preferably covered with an adhesive, to which sensor housing 12, and then brackets 74′, 74″ can be adhered. Additionally, and not shown, a release liner may be placed over the entire structure for storage prior to use.

Preferably, at least one bracket overlies the central portion of sensor housing 12, as shown in FIG. 5, to provide a centering or positioning point for the fingertip of a patient. By placing a finger at the bracket position, sensor housing 2 may be flexed into operative position for optimum trans-illumination of the finger.

The utilization of flexible straps, butterfly straps, adhesive tape, brackets, are generally known in the art, and one of ordinary skill in the art can contemplate other advantageous application structures without deviating from the intended scope of this invention. Such conventional devices, however, all still have the same drawback that, once utilized, the entire structure must be disposed of due to sanitary and patient acceptance concerns.

The present invention, on the other hand, may have portions extracted from used devices, and sanitized, so that remanufacturing is made possible. To that end, a user may sell a device, for example as shown in FIG. 3, to an end user for use. After use, the user may reacquire the device from the end user, and remove the unsanitary portions of the device. In the present invention, the user would remove the backing substrate 60 and butterfly strap 62 for disposal, leaving the sealed sensor housing 12, and wiring device 82. Since sensor housing 12 is sealed, and is manufactured from fluid-resistant materials, the entire remaining portion may be sanitized using known methods, and then reinserted into a new transillumination device 10, with a new backing substrate 60 and a new butterfly strap 62. Such a method can be used with any of the embodiments disclosed above.

The above method comprises an improvement over the prior art because it represents a clean and sanitary way to reduce the overall cost of manufacturing an otherwise disposable unit.

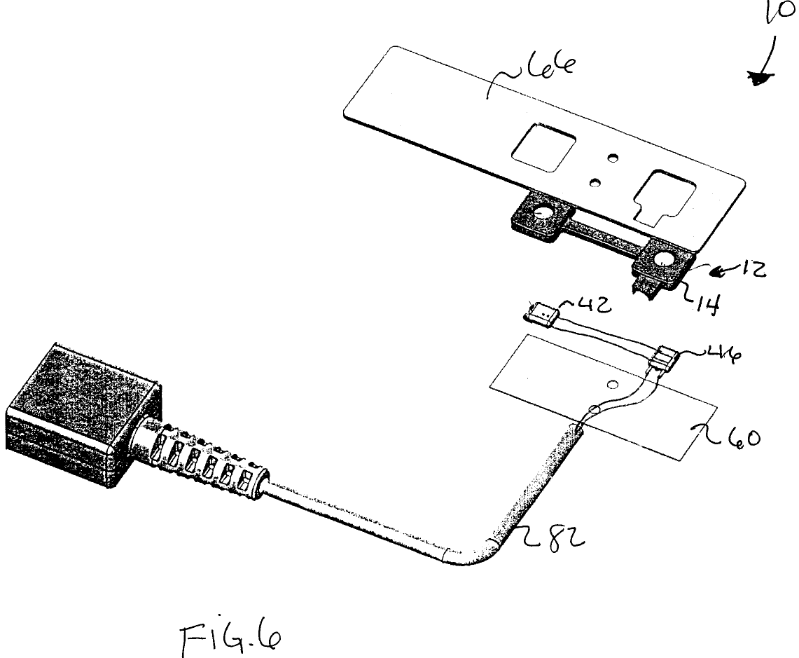

Although sensor housing 12 has been thusfar disclosed as being a sealed structure, it is possible to utilize the present invention with only top member 14 of sensor housing 12 alone. Thus, as shown in another alternative embodiment in FIG. 6, sensor housing 12 can comprise top member 14 alone, with emitter 42 and detector 46 (connected to wiring device 82 via wiring or other electrical connection) being placed directly on backing substrate 60. Backing substrate 60 preferably includes adhesive thereon, such that emitter 42 and detector 46 can be adhered thereto, top member 14 can be adhered over those elements, and flexible strap 66 can secure all elements therebetween. Unlike the previous embodiments, sensor housing 12 is not a sealed enclosure on all sides, but portions of the device may still be recovered for remanufacturing.

The foregoing description merely explains and illustrates the invention and the invention is not limited thereto except insofar as the appended claims are so limited, as those skilled in the art that have the disclosure before them will be able to make modifications without departing from the scope of the invention.

1. A sensor housing for measuring light transmission across a tissue of a patient, comprising:
a unitarily constructed top member and bottom member joined at a crease point;
the top and bottom members being foldable towards each other at the crease point to, in turn, create an enclosure therebetween;
the top member having a first and a second aperture therein; and
the top and bottom members comprising a flexible material such that, when placed in operative position on the tissue of a patient, the sensor housing may be flexed so that the first and second apertures are in substantial optical alignment.

2. The sensor housing of claim 1, wherein the top and bottom members are constructed of a material comprising one or more of an opaque material, a low Shore hardness material, a material that will limit slippage adjacent a patient's tissue, and a malleable material that conforms to the shape of a patient's tissue.

3. The sensor housing of claim 1, additionally comprising means for sealing the enclosure against intrusion of one or more of foreign bodies, moisture and ambient light.

4. The sensor housing of claim 3, wherein the sealing means comprises a seal surrounding a periphery of the enclosure, the seal comprising one or more of an adhesive seal, an ultrasonic seal, and a welded seal.

5. The sensor housing of claim 1, wherein the enclosure comprises an emitter housing, a detector housing and a wiring conduit therebetween.

6. The sensor housing of claim 5, wherein the emitter housing encloses an emitter therein, and the detector housing encloses a detector therein, wherein the emitter and detector are electrically connected through an electrical connection means through the wiring conduit.

7. The sensor housing of claim 6, wherein the enclosure has a top portion adjacent the top member and a bottom portion adjacent the bottom member, and a thickness therebetween, the sensor housing having at least one of the emitter being placed proximate the top portion of the enclosure, and the detector being placed proximate the bottom portion of the enclosure.

8. The sensor housing of claim 6, wherein the detector comprises a rear side, and has a coating of conductive material on the rear side of the detector.

9. The sensor housing of claim 8, wherein the conductive material comprises copper.

10. The sensor housing of claim 1, wherein the top member includes a top surface, wherein the top surface comprises a raised portion that approximates the curvature of a patient's tissue.

11. A trans-illumination device comprising:
a sensor housing as described in claim 1;
a backing substrate to which the sensor housing is affixed;
means for affixing the sensor housing to the backing substrate; and
means for attaching the trans-illumination device to a patient.

12. The device of claim 11, wherein the affixing means comprising a flexible strap having at least one aperture, and the sensor housing including an emitter head and a detector head configured to be inserted into the at least one aperture, and the flexible strap overlays and is attached to the sensor housing and the backing substrate to, in turn, affix the sensor housing to the backing substrate.

13. The device of claim 12, wherein the attaching means comprises an adhesive associated with one or more of the flexible strap, the sensor housing, and the backing substrate.

14. The device of claim 12, wherein the attaching means comprises a Velcro® strap associated with the flexible strap.

15. The device of claim 12, wherein at least one of the emitter head and detector head comprises a flexible flange, wherein the flange facilitates attachment to the flexible strap.

16. The device of claim 12, wherein at least one of the emitter head and detector head extends above a top side of the flexible strap.

17. The device of claim 11, further comprising a wiring device electrically connected to the sensor housing, wherein the affixing means comprises at least one bracket overlaying one or more of the sensor housing and the wiring device, and attached to the backing substrate.

18. The device of claim 17, wherein the attaching means comprises an adhesive associated with one or more of the bracket, the sensor housing, and the backing substrate.

19. The device of claim 17, wherein the attaching means comprises a Velcro® strap associated with the backing substrate.

20. The device of claim 17, wherein the sensor housing extends above a top side of the bracket.

21. The device of claim 17, wherein the bracket comprises means for aligning a finger of a patient with the trans-illuminating device.

22. A method of manufacturing a sensor housing for a transillumination device, comprising the steps of:
molding a top member and a bottom member from a unitary piece of material, wherein the top and bottom members include a crease point, and the top member includes a first and a second aperture;
inserting an emitter and a detector into at least one of the top and bottom members so that they are in substantial optical alignment with the first and second aperture respectively;
connecting the emitter and detector together with an electrical connection;
securing the emitter and detector in the at least one top and bottom member; and
folding the top and bottom members at the crease point to, in turn, form an enclosure therebetween.

23. The method according to claim 22, further including the step of sealing the enclosure around a periphery of the top and bottom members.

24. The method according to claim 23, wherein the step of sealing comprises at least one of the steps of ultrasonic sealing, applying an adhesive seal, and heat sealing.

25. A method for remanufacturing an otherwise disposable transillumination device, the method comprising the steps of:
acquiring an otherwise disposable device, the device comprising a sealed sensor housing according to claim 1, connected to a wiring device, and a means for attaching the sensor housing to a patient;
removing the attaching means from the disposable device;
sanitizing or sterilizing the sealed sensor housing and the wiring device; and
reassociating the sealed sensor housing with a new attaching means to, in turn, facilitate the use of the device on a patient

26. The method according to claim 25, wherein the attaching means comprises a backing substrate to which the sensor housing is affixed, and a flexible strap overlaying and affixed to the sensor housing and the backing substrate.

27. The method according to claim 25, wherein the attaching means comprises a backing substrate to which the sensor housing is affixed, and at least one bracket overlaying and affixed to at least one of the sensor housing and the wiring device and the backing substrate.

28. A method of decreasing the cost of an otherwise disposable medical unit, comprising the steps of:
acquiring an otherwise disposable medical device, wherein the medical device includes a sensor housing according to claim 1, a backing substrate to which the sensor housing is affixed, means for affixing the sensor housing to the backing substrate; and means for attaching the trans-illumination device to a patient;
removing the sensor housing, and disposing of the remainder of the medical device;
sanitizing or sterilizing the sensor housing; and
reinserting the sensor housing into a new otherwise disposable medical device to, in turn, reduce overall costs for remanufacturing the device.
